(19) United States
(12) Patent Application Publication
(10) Pub. No.: US 2025/0115832 A1
(43) Pub. Date: Apr. 10, 2025

(54) IMPROVEMENTS IN OR RELATING TO ORGANIC COMPOUNDS

(71) Applicant: Givaudan SA, VERNIER (CH)

(72) Inventor: Emmanuel AUSSANT, Argenteuil (FR)

(21) Appl. No.: 18/293,714

(22) PCT Filed: Aug. 9, 2022
(86) PCT No.: PCT/EP2022/072302

§ 371 Date: Jan. 30, 2024

(30) Foreign Application Priority Data
Aug. 10, 2021 (GB) 2111473.1

(51) Int. Cl. C11D 3/50; C11D 1/62; C11D 3/20; C11D 3/37

(52) U.S. Cl. CPC C11D 3/505; C11D 1/62; C11D 3/2082; C11D 3/37; C11D 2111/12

(57) ABSTRACT

Disclosed is an encapsulated composition comprising at least one core-shell microcapsule. The at least one core-shell microcapsule comprises a core containing at least one perfume ingredient and a shell surrounding the core. The shell comprises a thermosetting resin formed by reaction of a polyfunctional amine comprising at least one amino group with at least one polyfunctional isocyanate. The shell further comprises a cationic polymer comprising quaternary ammonium groups. The shell further comprises a polymeric stabilizer comprising fully or partially dissociated carboxylic acid groups. The nominal molar ratio of the amino groups and the quaternary ammonium groups to the carboxylic acid groups is from 0.9 to 1.1, preferably from 0.95 to 1.08, more preferably from 1.00 to 1.06.

The present invention is concerned with an encapsulated composition comprising at least one core-shell microcapsule. The invention also relates to a method for obtaining an encapsulated composition as well as to a use of such an encapsulated composition to obtain a consumer product.

The demand for encapsulated perfumery across all categories of consumer products, including personal care, household care, and particularly laundry care products continues to grow. As a result, formulators are required to incorporate perfume-containing microcapsules in ever more diverse product types and in ever more challenging (e.g. aggressive or extractive) media.

This growing customer demand reflects the increasing importance of scent to consumers of personal care, household care and fabric care products. Scent provides an olfactive cue that creates an impression amongst consumers of freshness and cleanliness, which in turn reinforces consumer confidence in the efficacy of such products.

There are many time points during which a consumer will interact with a consumer product before, during and after a cleaning or treatment experience. In the case of laundry products, by way of example, interaction points during a laundry experience will include the freshness experience a consumer receives when opening a container of a fabric care product; or when opening a washing machine or drier after washing or drying laundry; or the experience of freshness associated with ironing, folding or generally handling freshly laundered clothes or linen. If a laundry product can delight consumers during these moments of interaction, it can help transform a laborious chore into a more pleasant experience, and create moments of pleasure that promote brand loyalty and encourage product re-purchase.

The technique of microencapsulation offers the possibility to control the spatio-temporal release of fragrance during a cleaning or treatment experience, thus helping to create the aforementioned consumer benefits.

A wide variety of encapsulating media and perfume ingredients suitable for the preparation of encapsulated perfume compositions has been proposed in the art.

Encapsulating media proposed in the art include synthetic resins made from polyamides, polyureas, polyurethanes, polyacrylates, melamine-derived resins, or mixtures thereof; or naturally-occurring polymers, such as gelatin or polysaccharides.

As for suitable core materials, in principal, all perfume ingredients on a perfumer's palette can be incorporated to some extent into a core-shell microcapsule. However, it is generally accepted that certain physicochemical characteristics of a perfume ingredient, most notably its clogP, will influence whether and to what extent it can be encapsulated, and once encapsulated, its propensity to remain in the core without substantial leakage during storage. In the hands of the skilled formulator, the judicious selection of both the shell and core materials can result in microencapsulated perfumery that is stable in many consumer products and which is able to modulate the release of fragrance over time.

However, even when using relatively stable shell chemistries in combination with a well-designed perfume formulation in the core, the formulator is faced with the problem of colloidal stability of the microcapsules in various target products, in particular in products having different pH. Colloidally unstable microcapsules have the tendency to aggregate and ultimately to phase separate from the product in which they were dispersed. Furthermore, colloidal instability may lead to undesired change of flow properties, such as viscosity, of the product.

WO 2019/121736 A1 discloses core-shell microcapsules comprising a polymeric stabilizer combining a polymeric surfactant and an aminosilane, and a variety of shell-forming materials selected from the group of consisting of monomers, pre-polymers and pre-condensates. While these microcapsules provide the desired properties in terms of stability with respect to fragrance leakage and fragrance release, they may suffer from colloidal instabilities, such as aggregation, in consumer products containing ionic surfactants.

Consequently, there remains a need for improved enapsulation technologies that are colloidally stable in various products over a broad pH range, while still being performant on treated surfaces, such as fabric and keratinous surfaces.

These problems are solved by the subject-matter of the independent claims.

In a first aspect, the present invention provides an encapsulated composition comprising at least one core-shell microcapsule. The at least one core-shell microcapsule comprises a core containing at least one perfume ingredient and a shell surrounding the core. The shell comprises a thermosetting resin formed by reaction of a polyfunctional amine comprising at least one amino group with at least one polyfunctional isocyanate. The shell further comprises a cationic polymer comprising quaternary ammonium groups, as well as a polymeric stabilizer comprising fully or partially dissociated carboxylic acid groups. The nominal molar ratio of the amino groups and the quaternary ammonium groups to the carboxylic acid groups is from 0.8 to 1.1, preferably from 0.9 to 1.08, more preferably from 1.00 to 1.06.

By “nominal molar ratio” is meant a molar ratio calculated based on the total amount of materials engaged in the system. In the context of the present invention, the nominal molar ratio of the amino groups and the quaternary ammonium groups to the carboxylic acid groups is calculated based on the total amount of amino groups, quaternary ammonium groups and carboxylic groups present in the system.

In particular, the molar ratio mentioned hereinabove is calculated by calculating the total number of amino group equivalents present in the composition, including primary, secondary, ternary and quaternized amines, and the total number of carboxylic group equivalents. For polyethyleneimine (see below), the calculation may be simplified by considering the average number of ethyleneimine involved per polyethyleneimine chains, i.e. by dividing the average molecular weight of the polymer by the molecular weight of the ethyleneimine.

In addressing the problems of the prior art, the applicant has discovered that microcapsules having an optimal balance between amino and quaternary ammonium groups, on one hand, and fully or partially dissociated carboxylic acid groups, on the other hand, showed also an optimal colloidal stability in products having a broad range of compositions, in particular a broad range of surfactants and pH conditions, without showing any aggregation or phase separation.

Furthermore, the applicant has found that if this ratio is lower than 0.8, the microcapsules have a tendency to form aggregates in the presence of cationic surfactants over a broad range of pH, whereas, if this ratio is larger than 1.1, then the microcapsules have a tendency to form aggregates in the presence of anionic surfactants over a broad range of pH.

Furthermore, the applicant has surprisingly found that if the molar ratio of the amino groups and quaternary ammonium groups to the carboxylic acid groups is below 0.8, the volume-median diameter of the microcapsules dramatically increase, making them unsuitable for the sake of the invention.

The at least one polyfunctional isocyanate may be selected from organic isocyanates, in which an isocyanate group is bonded to an organic residue (R-N═C═O or R-NCO). In the context of the present invention, polyisocyanates (or polyfunctional isocyanates) are organic isocyanates with two or more (e.g. 3, 4, 5, etc.) isocyanate groups in a molecule. Suitable polyisocyanates are, for instance, aromatic, arylaliphatic, alicyclic or aliphatic.

Anionically modified polyisocyanates comprise at least two isocyanate groups and at least one functional group which is anionic or anionogenic. An “anionogenicfunctional group” is a group which can become anionic depending on the chemical environment, for instance the pH. Suitable anionic or anionogenic groups are, for instance, carboxylic acid groups, sulfonic acid groups, phosphonic acids groups and salts thereof.

In context of the present invention, an anionically modified polyisocyanate, in the following referred to as “anionicallymodified polyisocyanate (A)”, can comprise one or more sulfonic acid group or salts thereof. Suitable salts can be sodium, potassium or ammonium salts. Ammonium salts are preferred.

Preferably, anionically modified polyisocyanate (A) is obtained by reaction of a polyisocyanate with 2-(cyclohexylamino)-ethanesulfonic acid and/or 3-(cyclohexylamino)-propanesulfonic acid.

More preferably, anionically modified polyisocyanate (A) is obtained by reaction of a polyisocyanate with 2-(cyclohexylamino)-ethanesulfonic acid and/or 3-(cyclohexylamino)-propanesulfonic acid, wherein the polyisocyanate is selected from hexamethylene diisocyanate, tetramethylene diisocyanate, isophorone diisocyanate, dicyclohexylmethane-4,4′-diisocyanate, 2,4- and 2,6-toluylene diisocyanate and isomer mixtures thereof, diphenylmethane diisocyanates, biurets, allophanates and/or isocyanurates of the before-mentioned polyisocyanates.

Anionically modified polyisocyanate (A) can be selected in each case from anionically modified hexamethylene diisocyanate, isophorone diisocyanate, dicyclohexylmethane-4,4′-diisocyanate, the isocyanurate of hexamethylene diisocyanate and mixtures thereof.

Preferably, anionically modified polyisocyanate (A) has:
an average isocyanate functionality of at least 1.8,
a content of isocyanate groups (calculated as NCO; molecular weight=42) of 4.0 to 26.0 wt.-%,
a content of sulfonate groups (calculated as SO₃; molecular weight=80) of 0.1 to 7.7 wt.-% and
optionally a content of ethylene oxide units bonded within polyether chains (calculated as C₂H₂O; molecular weight=44) of 0 to 19.5 wt. %, wherein the polyether chains contain a statistical average of 5 to 55 ethylene oxide units.

In particular, anionically modified polyisocyanate (A) can be selected from an anionically modified hexamethylene diisocyanate, an anionically modified hexamethylene diisocyanate, an anionically modified isocyanurate of hexamethylene diisocyanate and mixtures thereof.

In a particularly preferred embodiment, anionically modified polyisocyanate (A) can be according to Formula I.

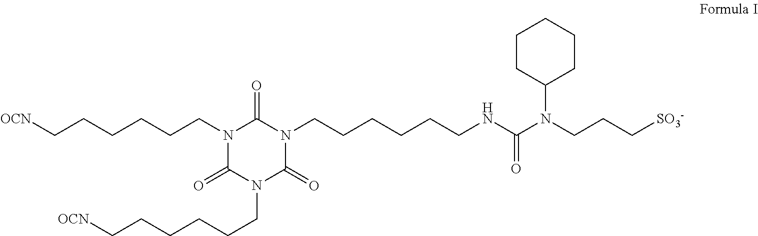

Formula I shows a commercially available anionically modified polyisocyanate, which is a modified isocyanurate of hexamethylene diisocyanate, sold by Covestro under the trademark Bayhydur© XP2547.

In particular embodiments of the present invention, a non-ionic polyisocyanate, herein referred to as “polyisocyanate (B)” can be used.

The non-ionic polyisocyanate (B) can be selected from the group consisting of 1,6-diisocyanatohexane, 1,5-diisocyanato-2-methylpentane, 1,5-diisocyanato-3-methylpentane, 1,4-diisocyanato-2,3-dimethylbutane, 2-ethyl-1,4-diisocyanatobutane, 1,5-diisocyanatopentane, 1,4-diisocyanatobutane, 1,3-diisocyanatopropane, 1,10-diisocyanatodecane, 1,2-diisocyanatocyclo-butane, bis(4-isocyanatocyclohexyl)methane, 3,3,5-trimethyl-5-isocyanatomethyl-1-isocyanato-cyclohexane, isophorone diisocyanate (IPDI), hexamethylene 1,6 diisocyanate (HDI), hydrogenated 4,4 diphenyl methane diisocyanate (HMDI).

Polyisocyanate (B) can also be a non-ionic oligomer based on the above-mentioned isocyanate monomers, such as for example the homopolymer of 1,6-diisocyanatohexane. All those monomers and oligomers are sold under the trade name Desmodur by Covestro AG.

Preferably, non-ionic polyisocyanate (B) is selected from hexamethylene diisocyanate, tetramethylene diisocyanate, dicyclohexylmethane-4,4′-diisocyanate, 2,4- and 2,6 toluylene diisocyanate and isomer mixtures thereof, 2,4′- and 4,4′-diphenylmethane diisocyanate and isomer mixtures thereof, xylylene diisocyanate (for example Desmodur quix 175 sold by Covestro), optionally as a trimethylolpropane (TMP) adduct (for example commercially available under the trademark Takenate D-110N), the biurets, allophanates and/or isocyanurates of the afore-mentioned polyisocyanates or mixtures thereof.

A preferred commercially available non-ionic polyisocyanate (B) is dicyclohexylmethane diisocyanate, in particular sold by Covestro AG under the trademark Desmodur W1.

A preferred commercially available non-ionic polyisocyanate (B) is hexamethylene diisocyanate, in particular sold by Covestro AG under the trademark Desmodur N3200.

A preferred commercially available non-ionic polyisocyanate (B) is isophorone diisocyanate, in particular sold by Covestro AG under the trademark Desmodur Z.

These polyisocyanates have the advantage of being non-aromatic and therefore more sustainable and less prone to oxidation, while still having high reactivity with polyamines and suitable molecular structure for the formation of impervious encapsulating resins.

In context of the present invention, the at least one polyfunctional isocyanate can comprise, preferably consist of, an anionically modified polyisocyanate (A) and a non-ionic polyisocyanate (B).

In a preferred embodiment of the present invention, the at least one polyfunctional isocyanate comprises, preferably consists of, an anionically modified polyisocyanate (A), selected from anionically modified hexamethylene diisocyanate, anionically modified isophorone diisocyanate, anionically modified dicyclohexylmethane-4,4′-diisocyanate, the anionically modified isocyanurate of hexamethylene diisocyanate and mixtures thereof and a non-ionic polyisocyanate (B), selected from hexamethylene diisocyanate, isophorone diisocyanate, dicyclohexylmethane-4,4′ diisocyanate, the isocyanurate of hexamethylene diisocyanate and mixtures thereof.

The weight ratio of anionically modified polyisocyanate (A) to non-ionic polyisocyanate (B) can be in the range of from 0.05 to 0.5, preferably from 0.07 to 0.25. These weight ratios provide resins having the highest imperviousness and therefore the most suitable for encapsulation.

In context of the present invention, the term “polyfunctional amine comprising atleast one amino group” denotes amines that comprise at least two groups capable of reacting with NCO groups, wherein at least one of the groups capable of reacting with NCO groups is a primary or secondary amino group. When the polyfunctional amine contains only one primary or secondary amino group, it will contain one or more additional functional groups that are capable of reacting with NCO groups in a polymerisation reaction. The groups of the polyfunctional amines that are reactive toward NCO groups are preferably chosen from hydroxyl groups and primary or secondary amino groups. Reaction of NCO groups with amino groups leads to the formation of urea groups. Reaction of NCO groups with OH groups leads to the formation of urethane groups. However, the reaction with OH groups often requires a catalyst. The amount of polyfunctional amines, which is introduced, is usually in a molar excess relative to the stoichiometric amount needed to convert the free isocyanate groups.

The polyfunctional amine can be selected from diamines, triamines, tetramines, and higher order polyfunctional amines, aminoalcohols, melamines, urea, hydrazines, polymeric polyamines, and mixtures thereof.

Suitable diamines are, for example, 1,2-ethylenediamine, 1,3-propylenediamine, 1,4 diaminobutane, 1,5-diaminopentane, 1,6-diaminohexane, 1,3-diamino-1-methylpropane, 1,4-diaminocyclohexane, piperazin or mixtures thereof.

Suitable amino alcohols are, for example, 2-aminoethanol, 2-(N-methylamino)ethanol, 3-aminopropanol, 4-aminobutanol, 1-ethylaminobutan-2-ol, 2-amino-2-methyl-1-propanol, 4 methyl-4-aminopentan-2-ol or mixtures thereof.

Suitable polymeric polyamines are in principle linear or branched polymers that have at least two primary or secondary amino groups. Additionally, these polymers can have tertiary amino groups in the polymer chain.

The polymeric polyamine is preferably selected from polyalkyleneamines, polyvinylamines, polyetheramines and mixtures thereof. More preferably, the polymeric polyamine is selected from polyalkyleneimines, in particular polyethyleneimines.

Preference is given to polymeric polyamines having a weight-average molecular weight of at least 300 g/mol. More preferred are polymeric polyamines having a weight-average molecular weight of from 500 to 2 000 000 g/mol, in particular from 700 to 1 000 000 g/mol, even more particularly from 800 to 500 000 g/mol.

In a preferred embodiment, the polyfunctional amine comprises, preferably consists of, at least one polyethyleneimine.

Polyethyleneimines may be short chain polyethyleneimines with the general formula H₂N(CH₂CH₂NH)ₙH, wherein n is an integer >1 (n=2: diethylenetriamine; n=3: triethylenetetramine; n=4: tetraethylenepentamine). These are sometimes called polyethyleneamines or polyalkylenepolyamines. Polyethyleneimines may also be long chain polyethyleneimines.

In the processes according to the present invention, polyethyleneimines with a molecular weight of at least 500 g/mol, preferably from 600 to 30 000 or 650 to 25 000 g/mol and in particular from 700 to 10 000 g/mol or 850 to 5000 g/mol, are preferably used.

The polyfunctional amine can be a polyethyleneimine containing the following repeat units

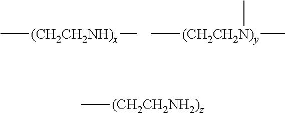

wherein
x is from 8 to 1500, preferably from 10 to 1000;
y is from 0 to 10, preferably from 0 to 5, especially 0;
z is 2+y.

With these polyethyleneimines good results can be achieved, in particular with respect to leakage in extractive media.

Preferred polyethyleneimines are linear polyethyleneimines, wherein x is from 8 to 1500, y is 0 and z is 2.

Preferred commercially available polyethylenimines are sold by BASF SE under the trademark Lupasol, particularly Lupasol G100.

Preferably, the weight ratio of polyfunctional amine to polyfunctional isocyanate is from 0.05 to 1, preferably from 0.1 to 0.5, still more preferably from 0.15 to 0.25. These weight ratios provide resins having high encapsulation efficiency.

The shell of the at least one microcapsule additionally comprises a polymeric stabilizer comprising fully or partially dissociated carboxylic acid groups. The polymeric stabilizer can be formed by combination of a polymeric emulsifier comprising the carboxylic acid groups with at least one aminosilane.

The polymeric stabilizers of the present invention stabilize the dispersed oil droplets, by ensuring that the droplets are prevented from coalescing and remain well suspended in the dispersing medium. In this way, the polymeric stabilizer helps to assist in the creation of a stable and versatile platform upon which different shell-forming chemistries can be deposited onto perfume oil droplets to form novel core-shell microcapsules.

Polymeric surfactants that are particularly suitable for the purpose of the present invention include copolymers, which are the reaction product of maleic anhydride and an olefinic monomer, such as ethylene, isobutylene or styrene. Examples of such copolymers include poly(ethylene-co-maleic anhydride), poly(isobutylene-co-maleic anhydride) and poly(styrene-co-maleic anhydride). A particularly preferred copolymer is poly(ethylene-co-maleic anhydride), a commercial grade of which is available under the trade name ZeMac E400. The maleic anhydride copolymer may be used singularly or alternatively combinations of maleic anhydride copolymers may be employed.

The maleic anhydride copolymer may be presented for use in the present invention in hydrolyzed form, whereupon the anhydride may be in the form of its free-acid, or its salt, or a mixture thereof.

If a maleic anhydride copolymer is used, it is particularly preferred if it is pre-hydrolyzed before being employed in the emulsification process. Hydrolysis can be achieved by dissolving the maleic anhydride in an aqueous medium, optionally at an elevated temperature, e.g. about 85 to 90° C., for an appropriate time interval. Typically 2 h is an appropriate time interval to affect hydrolysis. Once the polymer is dissolved under these conditions, the pH of the solution is typically below 3, which can be indicative that hydrolysis has taken place. Furthermore, infrared spectroscopic analysis reveals that the typical absorption bands corresponding to the anhydride group have vanished.

As stated hereinabove, the maleic anhydride copolymer in hydrolyzed form may be presented as its free acid, or its salt form, or a mixture of free acid and salt. The relative amounts of free acid and salt form will depend upon the pH of the aqueous medium. More particularly, the maleic anhydride copolymer is employed in aqueous solution at a pH of from about 2 to about 7, more particularly from about 4 to about 5, where the maleic anhydride copolymer exhibits optimal emulsifier properties.

The maleic anhydride copolymer in hydrolyzed form may be presented as a mixture of its free acid and salt form with monovalent counter-ions, such as lithium, sodium, potassium or ammonium counter-ions.

Alternatively, the polymeric surfactants may be selected from polysaccharides bearing carboxylic groups, including polysaccharide comprising uronic acid units, in particular hexuronic acid units. Polysaccharides having uronic acid units, in particular hexuronic acid units, are broadly available in nature.

The hexuronic acid units can be selected from the group consisting of galacturonic acid units, glucuronic acid units, in particular 4-O-methyl-glucuronic acid units, guluronic acid units and mannuronic acid units.

In particular embodiments of the present invention, the shell comprises a polymeric stabilizer that is formed by combination of the polymeric surfactant with at least one aminosilane, preferably by combination of 3-aminopropyltriethoxysilane with at least one of a poly(ethylene-co-maleic anhydride) and a poly(styrene-co-maleic anhydride).

In further embodiments of the present invention, the polymeric surfactant is selected from the group consisting of poly(acrylic acid) and copolymers thereof, poly(methacrylic acid) and copolymers thereof, hydrolyzed or partially hydrolyzed poly(maleic acid) and copolymers thereof, pectin, gum acacia, carboxymethylcellulose, alginate, hyaluronic acid, xanthan gum, gellan gum and their salts with monovalent alkaline metals.

The silane employed in the preparation of the polymeric stabilizer can be selected from a compound of Formula II

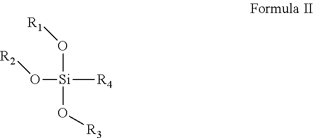

in which R₁, R₂ and R₃ are independently C₁-C₄ linear or branched alkyl or alkene, in particular methyl or ethyl, and R₄ is a C₁-C₁₂, preferably a C₁-C₄, linear or branched alkyl or alkene comprising a functional group. Particularly preferred are aminosilanes. The functional group can thus be an amine, in particular a primary, secondary or tertiary amine.

When the functional group is a primary amine, it can be a terminal primary amine. R₄ is then preferably a C₁-C₈, even more preferably a C₁-C₄, linear terminal primary aminoalkyl residue. Specific aminosilanes of this category are selected from the group consisting of aminomethyltriethoxysilane, 2-aminoethyltriethoxysilane, 3-aminopropyltriethoxysilane, 4-aminobutyltriethoxysilane, 5-aminopentyltriethoxysilane, 6-aminohexyltriethoxysilane, 7-aminohptyltriethoxysilane and 8-aminooctyltriethoxysilane, most preferably 3-aminopropyltriethoxysilane.

The aminosilane and the polymeric surfactant, which are combined to form the polymeric stabilizer, may be combined in widely varying amounts. However, it is preferred if the weight ratio of the polymeric surfactant, and more particularly the maleic anhydride copolymer, to the aminosilane is from 1 to 20, preferably from 1.5 to 10, still more preferably from 2.5 to 3.5.

The aminosilane can also be a bipodal aminosilane. By “bipodal aminosilane” is meant a molecule comprising at least one amino group and two residues, each of these residues bearing at least one alkoxysilane moiety.

In particular embodiments of the present invention, the at least one bipodal aminosilane has the Formula III.

(O-R⁴)_{(3-f)}(R³)_{f}Si-R²-X-R²-Si(O-R⁴)_{(3-f)}(R³)_{f} Formula III

In the above Formula III, X stands for —NR¹—, —NR¹—CH₂—NR¹—, —NR¹—CH₂—CH₂—NR¹—, —NR¹—CO—NR¹—, or

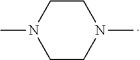

In the above Formula III, R¹ each independently stand for H, CH₃ or C₂Hs. R² each independently stand for a linear or branched alkylene group with 1 to 6 carbon atoms. R³ each independently stand for a linear or branched alkyl group with 1 to 4 carbon atoms. R⁴ each independently stand for H or for a linear or branched alkyl group with 1 to 4 carbon atoms. f stands for 0, 1 or 2.

Bipodal aminosilanes are particularly advantageous for forming stable oil-water interfaces, compared to conventional silanes. Examples of bipodal aminosilanes include, but are not limited to, bis(3-(triethoxysilyl)propyl)amine, N,N′-bis(3-(trimethoxysilyl)propyl)urea, bis(3-(methyldiethoxysilyl) propyl)amine, N,N′-bis(3-(trimethoxysilyl)propyl)ethane-1,2-diamine, bis(3-(methyldimethoxysilyl)propyl)-N-methylamine and N,N′-bis(3-(triethoxysilyl) propyl)piperazine.

The bipodal aminosilane can be a secondary aminosilane. Using a secondary bipodal aminosilane instead of primary aminosilane decreases the reactivity of the polymeric stabilizer with respect to electrophilic species, in particular aldehydes. Hence, benefit agents containing high levels of aldehydes may be encapsulated with a lower propensity for adverse interactions between core-forming and shell-forming materials.

The secondary bipodal aminosilane can be bis(3-(triethoxysilyl)propyl)amine. This particular secondary aminosilane has the advantage of releasing ethanol instead of more toxic and less desirable methanol during the polycondensation of the ethoxysilane groups.

In particular embodiments of the present invention, the polymeric stabilizer is formed by combination of a polymeric surfactant comprising the carboxylic acid groups with at least one aminosilane, preferably by combination of 3-aminopropyltriethoxysilane with at least one of a poly(ethylene-co-maleic anhydride) and a poly(styrene-co-maleic anhydride).

The weight ratio of the polymeric stabilizer to the thermosetting resin is preferably from 0.4 to 0.9, still more preferably from 0.45 to 0.75.

The shell of the microcapsules additionally comprises a cationic polymer comprising quaternary ammonium groups.

The cationic polymer can be derived from at least one monomer bearing quaternary ammonium functionality. In particular, the cationic monomer can be selected from the group consisting of quaternized dimethylaminoethyl acrylate (ADAME), quaternized dimethylaminoethyl methacrylate (MADAME), dimethyldiallyl ammonium chloride (DADMAC), acrylamidopropyltrimethylammonium chloride (APTAC) and methacrylamidopropyl-trimethylammonium chloride (MAPTAC).

The cationic polymer can further be additionally derived from a non-ionic monomer selected from the group consisting of water soluble vinyl monomers, more particularly acrylamide, methacrylamide, N-isopropylacrylamide, N,N-dimethylacrylamide, N-methylolacrylamide, N-vinylformamide, N-vinyl acetamide, N-vinylpyridine and/or N-vinylpyrrolidone.

The applicants have found that the quaternary ammonium groups are preferably provided by a cationic polymer, more preferably by a cationic copolymer, still more preferably a cationic block-copolymer comprising co-monomers that are not cationic. Without being bound by theory, the applicant believes that cationic polymers have the advantage of (i) extending in the aqueous wash or rinse liquor and (ii) maximizing the number of contact with the substrate, insuring thereby better deposition and substantivity of the microcapsules on this substrate. Furthermore, cationic copolymers comprising co-monomers that are not cationic have the advantage over cationic homopolymers to be more flexible and therefore more suitable for maximizing polymer-microcapsule and polymer-substrate interactions.

In preferred embodiments, the cationic polymer is a block co-polymer comprising quaternary ammonium groups and at least one co-monomer that is not cationic. Block copolymers having both types of monomers organized in distinct sequences are expected to provide an optimal balance between chain extension in the aqueous medium and anchoring on the substrate.

In particular embodiments of the present invention, the cationic polymer is selected from the group consisting of polyquaternium-2 (poly(bis[2-chloroethyl]ether-alt-1,3-bis[3-(dimethylamino)propyl]urea) copolymer), polyquaternium-4 (hydroxyethyl cellulose dimethyl diallylammonium chloride copolymer), polyquaternium-5 (poly(acrylamide-b-methacrylyloxyethyltrimethyl ammonium methosulfate) copolymer), polyquaternium-6 (poly(diallyldimethylammonium chloride) homopolymer), polyquaternium-7 (poly(acrylamide-co-diallyldimethyl-ammonium chloride) copolymer), polyquaternium-9 (poly(meth-acrylyloxyethyltrimethyl ammonium bromide) homopolymer), polyquaternium-10 (hydroxyethyl cellulose 2-hydroxyethyltrimethylammonium chloride copolymer), polyquaternium-11 (poly(vinylpyrrolidone-co-dimethylaminoethyl methacrylate) copolymer), polyquaternium-12 (poly(ethylmethycralate-co-abietylmethacrylate-co-methacrylyloxyethyltrimethyl ammonium dimethylsulfate) terpolymer), polyquaternium-13 (poly(ethylmethycralate-co-oleylmethacrylate-co-methacrylyloxyethyltrimethyl ammonium dimethylsulfate) terpolymer), polyquaternium-14 (poly(methacryloyloxy)-ethyl]-N,N,N-trimethylammonium methosulfate) homopolymer), polyquaternium-15 (poly(acrylamide-co-methacrylyloxyethyltrimethyl ammonium chloride) copolymer), polyquaternium-16 (poly(vinylpyrrolidone-co-vinylimidazanium chloride) terpolymer), polyquaternium-17, (adipic acid, dimethylaminopropylamine and dichloroethylether terpolymer), polyquaternium-18 (azelaic acid, dimethylaminopropylamine and dichloroethylether terpolymer), polyquaternium-19 (quaternized copolymer of poly(vinylalcohol) and 2,3-epoxy-propylamine copolymer), polyquaternium-22 (poly(acrylic acid-co-dimethyldiallyl ammonium chloride) copolymer), polyquaternium-28 (poly(vinylpyrrolidone-co-methacylamidopropyltrimethyl ammonium chloride) copolymer), polyquaternium-29 (chitosan modified with 2,3-dihydroxypropyl-2-hydroxy-3-(trimethylammonio)propyl ether, chloride), polyquaternium-32 (poly(acrylamide-co-methacryloyloxyethyltrimethyl ammonium chloride) copolymer), polyquaternium-33 (poly(acrylamide-co-acryloyloxyethyltrimethyl ammonium chloride) copolymer), polyquaternium-34 (1,3-dibromopropane and N,N-diethyl-N′,N′-dimethyl-1,3-propanediamine copolymer), polyquaternium-35 (poly(methacryloyloxyethyltrimethylammonium-co-methacryloyloxyethyldimethylacetylammonium methosulfate) copolymer), polyquaternium-36 (poly(butyl methacrylate-co-methacryloyloxyethyldimethylamine-co-methacryloyloxyethyl-trimethylammonium dimethylsulphate) terpolymer), polyquaternium-37 (poly(methacryloyloxyethyltrimethyl ammonium chloride) homopolymer), polyquaternium-39 (poly(acrylic acid-co-acrylamide-co-diallyl-dimethylammonium chloride) terpolymer), polyquaternium-42 (poly[oxyethylene(dimethylimino)ethylene (dimethylimino)ethylene dichloride) copolymer), polyquaternium-43 (poly(acrylamide-co-acrylamidopropyltrimethyl ammonium chloride-co-2-amidopropylacrylamide sulfonate-co-dimethylaminopropylamine) copolymer), polyquaternium-44 (poly(vinylpyrrolidone-co-imidazolinium) copolymer), polyquaternium-45, (poly([N-methyl-N-ethoxyglycine]-methacrylate-co-methacryloyloxyethyl-trimethyl ammonium dimethylsulfate) copolymer), polyquaternium-46 (poly(vinylcaprolactam-co-vinylpyrrolidone-co-vinylimidazolium methosulfate) terpolymer) and polyquaternium-47 (poly(acrylic acid-co-methacrylamidopropyl trimethylammonium chloride-co-methyl acrylate) terpolymer). Preferably, the cationic polymer is selected from polyquaternium-22 (poly(acrylic acid-co-dimethyldiallyl ammonium chloride) copolymer) and polyquaternium-39 (poly(acrylic acid-co-acrylamide-co-diallyl-dimethylammonium chloride) terpolymer).

Examples of commercially available polyquaterniums are available under the name of Merquat, ex Merck, such as Merquat 100 (polyquaternium 6), Merquat 740 (polyquaternium 7), Merquat 281 (polyquaternium 22) and Merquat 2001 (polyquaternium 47).

In preferred embodiments, the cationic polymer has an average molecular weight that is less than 500′000 g/mol, preferably less than 250′000 g/mol, still more preferably lower than 100′000 g/mol, for example 75′000 g/mol or less, or 50′000 g/mol or less. However, it is also preferred that the average molecular weight of the cationic polymer is higher than 5′000 g/mol, more preferably higher than 10′000 g/mol. Cationic polymers that have a higher average molecular weight may increase the viscosity of both microcapsule slurry and consumer product comprising the microcapsules. If the cationic polymer is too low, then the propensity of the copolymer to colloidally stabilize the microcapsule may decrease.

In addition to the polymer stabilizer, the fully or partially dissociated carboxylic acid groups may be provided also by cationic polymers mentioned hereinabove comprising (meth)acrylic acid co-monomers.

In particular embodiments, the level of shell material in the at least one core-shell microcapsule is from 2 wt.-% to 25 wt.-%, preferably from 5 wt.-% to 20 wt.-%, more preferably from 10 wt.-% to 15 wt.-%, based on the total weight of the at least one core-shell microcapsule.

In particular embodiments, a plurality of microcapsules according to the present invention has a volume-median diameter Dv(50) of from 1 to 50 μm, preferablyfrom 5 to 35 μm, still more preferably from 8 to 20 μm. Microcapsules having a diameter larger than 50 μm may have the disadvantage of being visible in the product. Furthermore, at constant microcapsule volume, the number of large microcapsules may not be sufficient to insure homogeneous dispersion of microcapsules in the product or homogeneous deposition of microcapsules on the substrate. Conversely, microcapsules that are too small have a high surface to volume ratio that makes them less stable with respect to leakage to the encapsulated perfume ingredients in the product base.

The microcapsules according to the present invention can be present in the form of a slurry, wherein the core-shell microcapsules are dispersed or suspended in an aqueous phase.

In particular embodiments, the slurry has a solid content of from 20 wt.-% to 60 wt.-%, preferably from 35 wt.-% to 45 wt.-%. The solid content of the slurry is typically measured by using a thermo-balance operating at 120° C. The solid content, expressed as weight percentage of the initial slurry deposited on the balance is taken at the point where the drying-induced rate of weight change had dropped below 0.1%/min.

In particular embodiments of the present invention, the perfume ingredients are selected from the group consisting of ACETYL ISOEUGENOL ((E)-2-methoxy-4-(prop-1-en-1-yl)phenyl acetate); ADOXAL (2,6,10-trimethylundec-9-enal); AGRUMEX (2-(tert-butyl)cyclohexyl acetate); ALDEHYDE C 10 DECYLIC (decanal); ALDEHYDE C 11 MOA (2-methyldecanal); ALDEHYDE C 11 UNDECYLENIC (undec-10-enal); ALDEHYDE C 110 UNDECYLIC (undecanal); ALDEHYDE C 12 LAURIC (dodecanal); ALDEHYDE C 12 MNA PURE (2-methylundecanal); ALDEHYDE C 8 OCTYLIC (octanal); ALDEHYDE C 9 ISONONYLIC (3,5,5-trimethylhexanal); ALDEHYDE C 9 NONYLIC FOOD GRADE (nonanal); ALDEHYDE C 90 NONENYLIC ((E)-non-2-enal); ALDEHYDE ISO C 11 ((E)-undec-9-enal); ALDEHYDE MANDARINE ((E)-dodec-2-enal); ALLYL AMYL GLYCOLATE (prop-2-enyl 2-(3-methylbutoxy)acetate); ALLYL CAPROATE (prop-2-enyl hexanoate); ALLYL CYCLOHEXYL PROPIONATE (prop-2-enyl 3-cyclohexylpropanoate); ALLYL OENANTHATE (prop-2-enyl heptanoate); AMBER CORE1-((2-(tert-butyl)cyclohexyl)oxy)butan-2-oIAMBERKETAL (3,8,8,11a-tetramethyldodecahydro-1H-3,5a-epoxynaphtho[2,1-c]oxepine); AMBERMAX (1,3,4,5,6,7-hexahydro-.beta.,1,1,5,5-pentamethyl-2H-2,4a-Methanonaphthalene-8-ethanol); AMBRETTOLIDE ((Z)-oxacycloheptadec-10-en-2-one); AMBROFIX ((3aR,5aS,9aS,9bR)-3a,6,6,9a-tetramethyl-2,4,5,5a,7,8,9,9b-octahydro-1H-benzo[e][1]benzofuran); AMYL BUTYRATE (pentyl butanoate); AMYL CINNAMIC ALDEHYDE ((Z)-2-benzylideneheptanal); AMYL SALICYLATE (pentyl 2-hydroxybenzoate); ANETHOLE SYNTHETIC ((E)-1-methoxy-4-(prop-1-en-1-yl)benzene); ANISYL ACETATE (4-methoxybenzyl acetate); APHERMATE (1-(3,3-dimethylcyclohexyl)ethyl formate); AUBEPINE PARA CRESOL (4-methoxybenzaldehyde); AURANTIOL ((E)-methyl 2-((7-hydroxy-3,7-dimethyloctylidene)amino)benzoate); BELAMBRE ((1R,2S,4R)-2′-isopropyl-1,7,7-trimethylspiro[bicyclo[2.2.1]heptane-2,4′-[1,3]dioxane]); BENZALDEHYDE (benzaldehyde); BENZYL ACETATE (benzyl acetate); BENZYL ACETONE (4-phenylbutan-2-one); BENZYL BENZOATE (benzyl benzoate); BENZYLSALICYLATE (benzyl 2-hydroxybenzoate); BERRYFLOR (ethyl 6-acetoxyhexanoate); BICYCLO NONALACTONE (octahydro-2H-chromen-2-one); BOISAMBRENE FORTE ((ethoxymethoxy)cyclododecane); BOISIRIS ((1S,2R,5R)-2-ethoxy-2,6,6-trimethyl-9-methylenebicyclo[3.3.1]nonane); BORNEOL CRYSTALS ((1S,2S,4S)-1,7,7-trimethylbicyclo[2.2.1]heptan-2-ol); BORNYL ACETATE ((2S,4S)-1,7,7-trimethylbicyclo[2.2.1]heptan-2-yl acetate); BOURGEONAL (3-(4-(tert-butyl)phenyl)propanal); BUTYL BUTYRO LACTATE (1-butoxy-1-oxopropan-2-yl butanoate); BUTYL CYCLOHEXYL ACETATE PARA (4-(tert-butyl)cyclohexyl acetate); BUTYL QUINOLINE SECONDARY (2-(2-methylpropyl)quinoline); CAMPHOR SYNTHETIC ((1S,4S)-1,7,7-trimethylbicyclo[2.2.1]heptan-2-one); CARVACROL (5-isopropyl-2-methylphenol); CARVONE LAEVO ((5R)-2-methyl-5-prop-1-en-2-ylcyclohex-2-en-1-one); CASHMERAN (1,1,2,3,3-pentamethyl-2,3,6,7-tetrahydro-1H-inden-4(5H)-one); CASSYRANE (5-tert-butyl-2-methyl-5-propyl-2H-furan); CEDRENE ((1S,8aR)-1,4,4,6-tetramethyl-2,3,3a,4,5,8-hexahydro-1H-5,8a-methanoazulene); CEDRYL ACETATE ((1S,6R,8aR)-1,4,4,6-tetramethyloctahydro-1H-5,8a-methanoazulen-6-yl acetate); CEDRYL METHYL ETHER ((1R,6S,8aS)-6-methoxy-1,4,4,6-tetramethyloctahydro-1H-5,8a-methanoazulene); CETONE V ((E)-1-(2,6,6-trimethylcyclohex-2-en-1-yl)hepta-1,6-dien-3-one); CINNAMIC ALCOHOL SYNTHETIC ((E)-3-phenylprop-2-en-1-ol); CINNAMIC ALDEHYDE ((2E)-3-phenylprop-2-enal); CINNAMYL ACETATE ((E)-3-phenylprop-2-en-1-yl acetate); CIS JASMONE ((Z)-3-methyl-2-(pent-2-en-1-yl)cyclopent-2-enone); CIS-3-HEXENOL ((Z)-hex-3-en-1-ol); CITRAL TECH ((E)-3,7-dimethylocta-2,6-dienal); CITRATHAL R ((Z)-1,1-diethoxy-3,7-dimethylocta-2,6-diene); CITRONELLAL (3,7-dimethyloct-6-enal); CITRONELLOL EXTRA (3,7-dimethyloct-6-en-1-ol); CITRONELLYL ACETATE (3,7-dimethyloct-6-en-1-yl acetate); CITRONELLYL FORMATE (3,7-dimethyloct-6-en-1-yl formate); CITRONELLYL NITRILE (3,7-dimethyloct-6-enenitrile); CLONAL (dodecanenitrile); CORANOL (4-cyclohexyl-2-methylbutan-2-ol); COSMONE ((Z)-3-methylcyclotetradec-5-enone); COUMARIN PURE CRYSTALS (2H-chromen-2-one); CRESYL ACETATE PARA ((4-methylphenyl) acetate); CRESYL METHYL ETHER PARA (1-methoxy-4-methylbenzene); CUMIN NITRILE (4-isopropylbenzonitrile); CYCLAL C (2,4-dimethylcyclohex-3-ene-1-carbaldehyde); CYCLAMEN ALDEHYDE EXTRA (3-(4-isopropylphenyl)-2-methylpropanal); CYCLOGALBANATE (allyl 2-(cyclohexyloxy)acetate); CYCLOHEXYL ETHYL ACETATE (2-cyclohexylethyl acetate); CYCLOHEXYL SALICYLATE (cyclohexyl 2-hydroxybenzoate); CYCLOMYRAL (8,8-dimethyl-1,2,3,4,5,6,7,8-octahydronaphthalene-2-carbaldehyde); CYMENE PARA (1-methyl-4-propan-2-ylbenzene); DAMASCENONE ((E)-1-(2,6,6-trimethylcyclohexa-1,3-dien-1-yl)but-2-en-1-one); DAMASCONE ALPHA ((E)-1-(2,6,6-trimethylcyclohex-2-en-1-yl)but-2-en-1-one); DAMASCONE DELTA (1-(2,6,6-trimethyl-1-cyclohex-3-enyl)but-2-en-1-one); DECALACTONE GAMMA (5-hexyloxolan-2-one); DECENAL-4-TRANS ((E)-dec-4-enal); DELPHONE (2-pentylcyclopentanone); DELTA-3 CARENE ((1S,6S)-3,7,7-trimethylbicyclo[4.1.0]hept-3-ene); DIHEXYL FUMARATE (dihexyl-but-2-enedioate); DIHYDRO ANETHOLE (1-methoxy-4-propylbenzene); DIHYDRO JASMONE (3-methyl-2-pentylcyclopent-2-enone); DIHYDRO MYRCENOL (2,6-dimethyloct-7-en-2-ol); DIMETHYL ANTHRANILATE (methyl 2-(methylamino)benzoate); DIMETHYL BENZYL CARBINOL (2-methyl-1-phenylpropan-2-ol); DIMETHYL BENZYL CARBINYL ACETATE (2-methyl-1-phenylpropan-2-yl acetate); DIMETHYL BENZYL CARBINYL BUTYRATE (2-methyl-1-phenylpropan-2-yl butanoate); DIMETHYL OCTENONE (4,7-dimethyloct-6-en-3-one); DIMETOL (2,6-dimethylheptan-2-ol); DIPENTENE (1-methyl-4-(prop-1-en-2-yl)cyclohex-1-ene); DIPHENYL OXIDE (oxydibenzene); DODECALACTONE DELTA (6-heptyltetrahydro-2H-pyran-2-one); DODECALACTONE GAMMA (5-octyloxolan-2-one); DODECENAL ((E)-dodec-2-enal); DUPICAL ((E)-4-((3aS,7aS)-hexahydro-1H-4,7-methanoinden-5(6H)-ylidene)butanal); EBANOL ((E)-3-methyl-5-(2,2,3-trimethylcyclopent-3-en-1-yl)pent-4-en-2-ol); ESTERLY (ethyl cyclohexyl carboxylate); ETHYL ACETATE (ethyl acetate); ETHYL ACETOACETATE (ethyl 3-oxobutanoate); ETHYL CINNAMATE (ethyl 3-phenylprop-2-enoate); ETHYL HEXANOATE (ethyl hexanoate); ETHYL LINALOOL ((E)-3,7-dimethylnona-1,6-dien-3-ol); ETHYL LINALYL ACETATE ((Z)-3,7-dimethylnona-1,6-dien-3-yl acetate); ETHYL MALTOL (2-ethyl-3-hydroxy-4H-pyran-4-one); ETHYL METHYL-2-BUTYRATE (ethyl 2-methylbutanoate); ETHYL OCTANOATE (ethyl octanoate); ETHYL OENANTHATE (ethyl heptanoate); ETHYL PHENYL GLYCIDATE (ethyl 3-phenyloxirane-2-carboxylate); ETHYL SAFRANATE (ethyl 2,6,6-trimethylcyclohexa-1,3-diene-1-carboxylate); ETHYL VANILLIN (3-ethoxy-4-hydroxybenzaldehyde); ETHYLENE BRASSYLATE (1,4-dioxacycloheptadecane-5,17-dione); EUCALYPTOL ((1s,4s)-1,3,3-trimethyl-2-oxabicyclo[2.2.2]octane); EUGENOL (4-allyl-2-methoxyphenol); EVERNYL (methyl 2,4-dihydroxy-3,6-dimethylbenzoate); FENCHYL ACETATE ((2S)-1,3,3-trimethylbicyclo[2.2.1]heptan-2-yl acetate); FENCHYL ALCOHOL ((1S,2R,4R)-1,3,3-trimethylbicyclo[2.2.1]heptan-2-ol); FENNALDEHYDE (3-(4-methoxyphenyl)-2-methylpropanal); FIXAMBRENE (3a,6,6,9a-tetramethyldodecahydronaphtho[2,1-b]furan); FIXOLIDE (1-(3,5,5,6,8,8-hexamethyl-5,6,7,8-tetrahydronaphthalen-2-yl)ethanone); FLORALOZONE (3-(4-ethylphenyl)-2,2-dimethylpropanal); FLORHYDRAL (3-(3-isopropylphenyl)butanal); FLORIDILE ((E)-undec-9-enenitrile); FLOROCYCLENE ((3aR,6S,7aS)-3a,4,5,6,7,7a-hexahydro-1H-4,7-methanoinden-6-yl propanoate); FLOROPAL (2,4,6-trimethyl-4-phenyl-1,3-dioxane); FLOROSA HC (tetrahydro-4-methyl-2-(2-methylpropyl)-2H-pyran-4-ol); FRESKOMENTHE (2-(sec-butyl)cyclohexanone); FRUCTONE (ethyl 2-(2-methyl-1,3-dioxolan-2-yl)acetate); FRUITATE ((3aS,4S,7R,7aS)-ethyl octahydro-1H-4,7-methanoindene-3a-carboxylate); FRUTONILE (2-methyldecanenitrile); GALBANONE PURE (1-(5,5-dimethylcyclohex-1-en-1-yl)pent-4-en-1-one); GARDENOL (1-phenylethyl acetate); GARDOCYCLENE ((3aR,6S,7aS)-3a,4,5,6,7,7a-hexahydro-1H-4,7-methanoinden-6-yl 2-methyl propanoate); GERANIOL ((E)-3,7-dimethylocta-2,6-dien-1-ol); GERANYL ACETATE ((E)-3,7-dimethylocta-2,6-dien-1-yl acetate); GERANYL CROTONATE ((E)-3,7-dimethylocta-2,6-dien-1-yl but-2-enoate); GERANYL ISOBUTYRATE ((E)-3,7-dimethylocta-2,6-dien-1-yl 2-methylpropanoate); GIVESCONE (ethyl 2-ethyl-6,6-dimethylcyclohex-2-enecarboxylate); HABANOLIDE ((E)-oxacyclohexadec-12-en-2-one); HEDIONE (methyl 3-oxo-2-pentylcyclopentaneacetate); HELIOTROPINE CRYSTALS (benzo[d][1,3]dioxole-5-carbaldehyde); HERBANATE ((2S)-ethyl 3-isopropylbicyclo[2.2.1]hept-5-ene-2-carboxylate); HEXENAL-2-TRANS ((E)-hex-2-enal); HEXENOL-3-CIS ((Z)-hex-3-en-1-ol); HEXENYL-3-CIS ACETATE ((Z)-hex-3-en-1-yl acetate); HEXENYL-3-CIS BUTYRATE ((Z)-hex-3-en-1-yl butanoate); HEXENYL-3-CIS ISOBUTYRATE ((Z)-hex-3-en-1-yl 2-methylpropanoate); HEXENYL-3-CIS SALICYLATE ((Z)-hex-3-en-1-yl 2-hydroxybenzoate); HEXYL ACETATE (hexyl acetate); HEXYL BENZOATE (hexyl benzoate); HEXYL BUTYRATE (hexyl butanoate); HEXYL CINNAMIC ALDEHYDE ((E)-2-benzylideneoctanal); HEXYL ISOBUTYRATE (hexyl 2-methylpropanoate); HEXYL SALICYLATE (hexyl 2-hydroxybenzoate); HYDROXYCITRONELLAL (7-hydroxy-3,7-dimethyloctanal); INDOFLOR (4,4a,5,9b-tetrahydroindeno[1,2-d][1,3]dioxine); INDOLE PURE (1H-indole); INDOLENE (8,8-di(1H-indol-3-yl)-2,6-dimethyloctan-2-ol); IONONE BETA ((E)-4-(2,6,6-trimethylcyclohex-1-en-1-yl)but-3-en-2-one); IRISANTHEME ((E)-3-methyl-4-(2,6,6-trimethylcyclohex-2-en-1-yl)but-3-en-2-one); IRISONE ALPHA ((E)-4-(2,6,6-trimethylcyclohex-2-en-1-yl)but-3-en-2-one); IRONE ALPHA ((E)-4-(2,5,6,6-tetramethylcyclohex-2-en-1-yl)but-3-en-2-one); ISO E SUPER (1-(2,3,8,8-tetramethyl-1,2,3,4,5,6,7,8-octahydronaphthalen-2-yl)ethanone); ISOAMYL ACETATE (3-methylbutyl acetate); ISOAMYL BUTYRATE (3-methylbutyl butanoate); ISOBUTYL METHOXY PYRAZINE (2-methylpropyl 3-methoxypyrazine); ISOCYCLOCITRAL (2,4,6-trimethylcyclohex-3-enecarbaldehyde); ISOEUGENOL ((E)-2-methoxy-4-(prop-1-en-1-yl)phenol); ISOJASMONE B 11 (2-hexylcyclopent-2-en-1-one); ISOMENTHONE DL (2-isopropyl-5-methylcyclohexanone); ISONONYL ACETATE (3,5,5-trimethylhexyl acetate); ISOPROPYL METHYL-2-BUTYRATE (isopropyl 2-methylbutanoate); ISOPROPYL QUINOLINE (6-isopropylquinoline); ISORALDEINE ((E)-3-methyl-4-(2,6,6-trimethylcyclohex-2-en-1-yl)but-3-en-2-one); JASMACYCLENE ((3aR,6S,7aS)-3a,4,5,6,7,7a-hexahydro-1H-4,7-methanoinden-6-yl acetate); JASMONE CIS ((Z)-3-methyl-2-(pent-2-en-1-yl)cyclopent-2-enone); JASMONYL (3-butyl-5-methyltetrahydro-2H-pyran-4-yl acetate); JASMOPYRANE FORTE (3-pentyltetrahydro-2H-pyran-4-yl acetate); JAVANOL ((1-methyl-2-((1,2,2-trimethylbicyclo[3.1.0]hexan-3-yl)methyl)cyclopropyl)methanol); KOAVONE ((Z)-3,4,5,6,6-pentamethylhept-3-en-2-one); LAITONE (8-isopropyl-1-oxaspiro[4.5]decan-2-one); LEAF ACETAL ((Z)-1-(1-ethoxyethoxy)hex-3-ene); LEMONILE ((2E,6Z)-3,7-dimethylnona-2,6-dienenitrile); LIFFAROME ((Z)-hex-3-en-1-yl methyl carbonate); LILIAL (3-(4-(tert-butyl)phenyl)-2-methylpropanal); #N/ALINALOOL (3,7-dimethylocta-1,6-dien-3-ol); LINALOOL OXIDE (2-(5-methyl-5-vinyltetrahydrofuran-2-yl)propan-2-ol); LINALYL ACETATE (3,7-dimethylocta-1,6-dien-3-yl acetate); MAHONIAL ((4E)-9-hydroxy-5,9-dimethyl-4-decenal); MALTOL (3-hydroxy-2-methyl-4H-pyran-4-one); MALTYL ISOBUTYRATE (2-methyl-4-oxo-4H-pyran-3-yl 2-methylpropanoate); MANZANATE (ethyl 2-methylpentanoate); MAYOL ((4-isopropylcyclohexyl)methanol); MEFROSOL (3-methyl-5-phenylpentan-1-ol); MELONAL (2,6-dimethylhept-5-enal); #N/A #N/AMERCAPTO-8-METHANE-3-ONE (mercapto-para-menthan-3-one); METHYL ANTHRANILATE (methyl 2-aminobenzoate); METHYL BENZOATE (methyl benzoate); METHYL CEDRYL KETONE (1-((1S,8aS)-1,4,4,6-tetramethyl-2,3,3a,4,5,8-hexahydro-1H-5,8a-methanoazulen-7-yl)ethanone); METHYL CINNAMATE (methyl 3-phenylprop-2-enoate); METHYL DIANTILIS (2-ethoxy-4-(methoxymethyl)phenol); METHYL DIHYDRO ISOJASMONATE (methyl 2-hexyl-3-oxocyclopentane-1-carboxylate); METHYL HEPTENONE PURE (6-methylhept-5-en-2-one); METHYL LAITONE (8-methyl-1-oxaspiro[4.5]decan-2-one); METHYL NONYL KETONE (undecan-2-one); METHYL OCTYNE CARBONATE (methyl non-2-ynoate); METHYL PAMPLEMOUSSE (6,6-dimethoxy-2,5,5-trimethylhex-2-ene); METHYL SALICYLATE (methyl 2-hydroxybenzoate); MUSCENONE ((Z)-3-methylcyclopentadec-5-enone); MYRALDENE (4-(4-methylpent-3-en-1-yl)cyclohex-3-enecarbaldehyde); MYRCENE (7-methyl-3-methyleneocta-1,6-diene); MYSTIKAL (2-methylundecanoic acid); NECTARYL (2-(2-(4-methylcyclohex-3-en-1-yl)propyl)cyclopentanone); NEOBERGAMATE FORTE (2-methyl-6-methyleneoct-7-en-2-yl acetate); NEOCASPIRENE EXTRA (10-isopropyl-2,7-dimethyl-1-oxaspiro[4.5]deca-3,6-diene); NEOFOLIONE ((E)-methyl non-2-enoate); NEROLEX ((2Z)-3,7-dimethylocta-2,6-dien-1-ol); NEROLIDOL ((Z)-3,7,11-trimethyldodeca-1,6,10-trien-3-ol); NEROLIDYLE ((Z)-3,7,11-trimethyldodeca-1,6,10-trien-3-yl acetate); NEROLINE CRYSTALS (2-ethoxynaphthalene); NEROLIONE (1-(3-methylbenzofuran-2-yl)ethanone); NERYL ACETATE ((Z)-3,7-dimethylocta-2,6-dien-1-yl acetate); NIRVANOLIDE ((E)-13-methyloxacyclopentadec-10-en-2-one); NONADIENAL ((2E,6Z)-nona-2,6-dienal); NONADIENOL-2,6 ((2Z,6E)-2,6-nonadien-1-ol); NONADYL (6,8-dimethylnonan-2-ol); NONALACTONE GAMMA (5-pentyloxolan-2-one); NONENAL-6-CIS ((Z)-non-6-enal); NONENOL-6-CIS ((Z)-non-6-en-1-ol); NOPYL ACETATE (2-(6,6-dimethylbicyclo[3.1.1]hept-2-en-2-yl)ethyl acetate); NYMPHEAL (3-(4-(2-methylpropyl)-2-methylphenyl)propanal); OCTALACTONE DELTA (6-propyltetrahydro-2H-pyran-2-one); METHYL HEXYL KETONE (octan-2-one); ORANGER CRYSTALS (1-(2-naphtalenyl)-ethanone); ORIVONE (4-(tert-pentyl)cyclohexanone); PANDANOL ((2-methoxyethyl)benzene); PARA TERT BUTYL CYCLOHEXYL ACETATE (4-(tert-butyl)cyclohexyl acetate); PARADISAMIDE (2-ethyl-N-methyl-N-(m-tolyl)butanamide); PEACH PURE (5-heptyldihydrofuran-2(3H)-one); PELARGENE (2-methyl-4-methylene-6-phenyltetrahydro-2H-pyran); PELARGOL (3,7-dimethyloctan-1-ol); PEONILE (2-cyclohexylidene-2-phenylacetonitrile); PETALIA (2-cyclohexylidene-2-(o-tolyl)acetonitrile); PHARAONE (2-cyclohexylhepta-1,6-dien-3-one); PHENOXY ETHYL ISOBUTYRATE (2-(phenoxy)ethyl 2-methylpropanoate); PHENYL ACETALDEHYDE (2-phenyl-ethanal); PHENYL ETHYLACETATE (2-phenylethyl acetate); PHENYL ETHYLALCOHOL (2-phenylethanol); PHENYL ETHYL ISOBUTYRATE (2-phenylethyl 2-methylpropanoate); PHENYL ETHYL PHENYL ACETATE (2-phenylethyl 2-phenylacetate); PHENYL PROPYL ALCOHOL (3-phenylpropan-1-ol); PINENE ALPHA (2,6,6-trimethylbicyclo[3.1.1]hept-2-ene); PINENE BETA (6,6-dimethyl-2-methylenebicyclo[3.1.1]heptane); PINOACETALDEHYDE (3-(6,6-dimethylbicyclo[3.1.1]hept-2-en-2-yl)propanal); PIVAROSE (2,2-dimethyl-2-pheylethyl propanoate); POMAROSE ((2E,5E)-5,6,7-trimethylocta-2,5-dien-4-one); POMELOL (2,4,7-Trimethyl-6-octen-1-ol); PRECYCLEMONE B (1-methyl-4-(4-methylpent-3-en-1-yl)cyclohex-3-enecarbaldehyde); PRENYL ACETATE (3-methylbut-2-en-1-yl acetate); PRUNOLIDE (5-pentyldihydrofuran-2(3H)-one); RADJANOL SUPER ((E)-2-ethyl-4-(2,2,3-trimethylcyclopent-3-en-1-yl)but-2-en-1-ol); RASPBERRY KETONE (4-(4-hydroxyphenyl)butan-2-one); RHUBAFURAN (2,4-dimethyl-4-phenyltetrahydrofuran); ROSACETOL (2,2,2-trichloro-1-phenylethyl acetate); ROSALVA (dec-9-en-1-ol); ROSE OXIDE (4-methyl-2-(2-methylprop-1-en-1-yl)tetrahydro-2H-pyran); ROSE OXIDE CO (4-methyl-2-(2-methylprop-1-en-1-yl)tetrahydro-2H-pyran); ROSYFOLIA (1-methyl-2-(5-methylhex-4-en-2-yl)cyclopropylmethanol); ROSYRANE SUPER (4-methyl-2-phenyl-3,6-dihydro-2H-pyran); SAFRALEINE (2,3,3-trimethyl-1-indanone); SAFRANAL (2,6,6-trimethylcyclohexa-1,3-dienecarbaldehyde); SANDALORE EXTRA (3-methyl-5-(2,2,3-trimethylcyclopent-3-en-1-yl)pentan-2-ol); SCENTAURUS CLEAN (ethyl (Z)-2-acetyl-4-methyltridec-2-enoate); SERENOLIDE (2-(1-(3,3-dimethylcyclohexyl)ethoxy)-2-methylpropyl cyclopropanecarboxylate); SILVANONE SUPRA (cyclopentadecanone, hexadecanolide); SILVIAL (2-methyl-3-[4-(2-methylpropyl)phenyl]propanal); SPIROGALBANONE (1-(spiro[4.5]dec-6-en-7-yl)pent-4-en-1-one); STEMONE ((E)-5-methylheptan-3-one oxime); STYRALLYL ACETATE (1-phenylethyl acetate); SUPER MUGUET ((E)-6-ethyl-3-methyloct-6-en-1-ol); SYLKOLIDE ((E)-2-((3,5-dimethylhex-3-en-2-yl)oxy)-2-methylpropyl cyclopropanecarboxylate); TERPINENE ALPHA (1-methyl-4-propan-2-ylcyclohexa-1,3-diene); TERPINENE GAMMA (1-methyl-4-propan-2-ylcyclohexa-1,4-diene); TERPINEOL (2-(4-methylcyclohex-3-en-1-yl)propan-2-ol); TERPINEOLALPHA (2-(4-methyl-1-cyclohex-3-enyl)propan-2-ol); TERPINEOL PURE (2-(4-methylcyclohex-3-en-1-yl)propan-2-ol); TERPINOLENE (1-methyl-4-(propan-2-ylidene)cyclohex-1-ene); TERPINYL ACETATE (2-(4-methyl-1-cyclohex-3-enyl)propan-2-yl acetate); TETRAHYDRO LINALOOL (3,7-dimethyloctan-3-ol); TETRAHYDRO MYRCENOL (2,6-dimethyloctan-2-ol); THIBETOLIDE (oxacyclohexadecan-2-one); THYMOL (2-isopropyl-5-methylphenol); TOSCANOL (1-(cyclopropylmethyl)-4-methoxybenzene); TRICYCLAL (2,4-dimethylcyclohex-3-enecarbaldehyde); TRIDECENE-2-NITRILE ((E)-tridec-2-enenitrile); TRIFERNAL (3-phenylbutanal); TROPIONAL (3-(benzo[d][1,3]dioxol-5-yl)-2-methylpropanal); TROPIONAL (3-(benzo[d][1,3]dioxol-5-yl)-2-methylpropanal); UNDECATRIENE ((3E,5Z)-undeca-1,3,5-triene); UNDECAVERTOL ((E)-4-methyldec-3-en-5-ol); VANILLIN (4-hydroxy-3-methoxybenzaldehyde); VELOUTONE (2,2,5-trimethyl-5-pentylcyclopentanone); VELVIONE ((Z)-cyclohexadec-5-enone); VIOLET NITRILE ((2E,6Z)-nona-2,6-dienenitrile); YARA YARA (2-methoxynaphtalene); ZINARINE (2-(2,4-dimethylcyclohexyl)pyridine; BOIS CEDRE ESS CHINE (cedar wood oil); EUCALYPTUS GLOBULUS ESS CHINA (eucalyptus oil); GALBANUM ESS (galbanum oil); GIROFLE FEUILLES ESS RECT MADAGASCAR (clove oil); LAVANDIN GROSSO OIL FRANCE ORPUR (lavandin oil); MANDARIN OIL WASHED COSMOS (mandarin oil); ORANGE TERPENES (orange terpenes); PATCHOULI ESS INDONESIE (patchouli oil); and YLANG ECO ESSENCE (ylang oil). These fragrance ingredients are particularly suitable for obtaining stable and performing microcapsules, owing to their favorable lipophilicity and olfactive performance.

A comprehensive list of fragrance ingredients that may also be encapsulated in accordance with the present invention may be found in the perfumery literature, for example “Perfume & Flavor Chemicals”, S. Arctander (Allured Publishing, 1994).

A second aspect of the present invention provides a method for obtaining an encapsulated composition, in particular a composition as described herein above. The method comprises the steps of:
a) Providing a core composition, comprising an aminosilane;
b) Providing an aqueous phase comprising at least one polymeric surfactant comprising fully or partially dissociated carboxylic acid groups;
c) Emulsifying the core composition provided in step a) into the aqueous phase provided in step b) to obtain an emulsion of core composition droplets dispersed in the aqueous phase;
d) Causing the aminosilane and the polymeric surfactant to form a polymeric stabilizer stabilizing the dispersed oil droplets;
e) Adding at least one polyfunctional isocyanate;
f) Adding at least one polyfunctional amine comprising at least one amino group; and
g) Causing the polyfunctional isocyanate and the polyfunctional amine to react and to form a shell around the core composition droplets, in order to form a slurry of microcapsules;
   wherein a cationic polymer comprising quaternary ammonium groups is added before, during or after step g), preferably before or during step g); and
   wherein the nominal molar ratio of the amino groups and the quaternary ammonium groups to the carboxylic acid groups is from 0.8 to 1.1, preferably from 0.9 to 1.08, more preferably from 1.00 to 1.06.

The appropriate stirring speed and geometry of the mixer can be selected in order to obtain the desired average droplet size and droplet size distribution. It is a characteristic of the present invention that the polymeric stabilizer has particularly high surfactant power and is able to promote the formation of dispersed oil droplets with desirable small droplet size and low polydispersity.

It is a characteristic of the process of the present invention that, in a one-liter vessel, equipped with a cross-beam stirrer with pitched beam, and having a stirrer diameter to reactor diameter of about 0.7 having, microcapsules can be formed having an average particle size D(50) of 30 microns or less, more particularly 20 microns or less, and with a polydispersity span of less than 1.5, more particularly less than 1.3, still more particularly less than 1.2, at a stirring speed of less than 1000 rpm, more particularly in the order of from about 100 to about 1000 rpm, still more particularly from about 500 to 700 rpm, for example 600 rpm using a turbine, a cross-beam stirrer with pitched beam, such as Mig stirrer, or the like. Preferably, a Mig stirrer is used operating at a speed of 600±50 rpm. The person skilled in the art will however easily understand that such stirring conditions may change depending on the size of the reactor and of the volume of the slurry, on the exact geometry of the stirrer on the ratio of the diameter of the stirrer to the diameter of the reactor diameter ratios. For example, for a Mig stirrer with stirrer to reactor diameter ratio from 0.5 to 0.9 and slurry volumes ranging from 0.5 to 8 tons, the preferable agitation speed in the context of the present invention is from 150 rpm to 50 rpm.

In the formation of the oil-in-water emulsion (steps a) to d)), the maleic anhydride copolymer is added to the aqueous external phase, and the aminosilane is admixed with the oil phase. Their separation is a process optimization consideration to control the rate of hydrolysis of the silane and to ensure that the silane and the maleic anhydride copolymer react at the oil-water interface in an optimal fashion to form the polymeric stabilizer in-situ. If the silane is allowed to hydrolyze too rapidly it is prone to self-condense. Employing the silane in the oil phase promotes its reaction with the polymeric surfactant at the oil-water interface, rather than undergoing self-condensation.

In order to provide optimal reaction conditions for the coupling of the aminosilane and the maleic anhydride, the pH of the mixture is raised to about 3.5 to 7, for example 4.5 or 6. This can be achieved by the addition of a suitable base. For this purpose, a dilute solution (20%) of ammonia is suitable, although other bases could be employed, such as dilute sodium hydroxide. The whole process can be carried out over a period of about 1 h to 3 h, more particularly 2 h±0.5 h, and at ambient, or slightly elevated temperature, e.g. 35±5° C. The polymeric stabilizer formed in-situ in this way becomes associated at the oil-water interface to form an at least partial layer around the oil droplets, stabilizing them and preventing coalescence.

In respect to step g), the formation of the shells around the droplets can be effected by heating. This can be achieved at a temperature of at least 50° C., preferably at least 60° C., more preferably in a range of from 65° C. to 90° C., in order to ensure sufficiently rapid reaction progress. It may be preferred to increase the temperature continuously or in stages (e.g. in each case by 5° C.) until the reaction is essentially complete. Afterwards, the dispersion may cool down to room temperature.

For formation of the shells around the droplets, the pH of the aqueous phase can be adjusted to a range of from 4 to 8, preferably from 5 to 7, for example around 6. The pH can be adjusted using an inorganic base, for example sodium hydroxide solution, or carbonate buffer salts.

The reaction time typically depends on the nature of the reactive wall-forming materials, the amount of said materials employed, and the temperature used. The period of time for the reaction is ranging from a few minutes to several hours. Usually, microcapsule formation is effected between ca. 60 minutes to 6 h or up to 8 h at the temperatures defined above.

In accordance with the process described herein, microcapsules can be obtained that exhibit good retention of their core contents, but are also rather frangible. In this way, the microcapsules are sufficiently robust that they exhibit low levels of leakage during storage even in extractive media, but in application a significant proportion can break relatively easily to release their core contents. This is particularly advantageous in encapsulated perfumery applications, and more particularly encapsulated perfumery in laundry applications.

Applicant believes, although does not intend to be bound by particular theory, that by operating within the process parameters described herein, including the selection of reagents, and in particular the control of the rate and/or duration of heating in the manner described, it is possible to control the reaction of the shell-forming monomers and create relatively thin and homogenous resinous shells, which resist leakage but which can break in response to only light or moderate shear force.

After formation of the microcapsules, the encapsulated composition can be cooled to room temperature. Preferably the cooling time is at least one hour, more particularly at least 2 h, for example 2.5 h±0.5 h. Slow cooling in this manner is believed that the resin is able to further arrange itself by annealing, which may also affect the homogeneity of the resin shells and therefore contribute to the properties of the microcapsules in application.

Before, during or after cooling, the encapsulated composition may be further processed. Further processing may include treatment of the composition with one or more anti-microbial preservatives, which preservatives are well known in the art. Further processing may also include the addition of a suspending aid, such as a hydrocolloid suspending aid to assist in the stable physical dispersion of the microcapsules and prevent any creaming or coalescence or whatsoever. Any additional adjuvants that may be desired, or conventional in the art, may also be added at this time.

The resultant encapsulated composition, presented in the form of a slurry of microcapsules suspended in an aqueous suspending medium, may be incorporated as such in a consumer product base. If desired, however, the slurry may be dehydrated to present the encapsulated composition in dry powder form. Dehydration of a microcapsule slurry is conventional, and may be carried out according techniques known in the art, such as spray-drying, evaporation or lyophilization. Typically, as is conventional in the art, dried microcapsules will be dispersed or suspended in a suitable powder, such as powdered silica or the like, which can act as a bulking agent, flow aid, or the like. Such suitable powder may be added to the encapsulated composition before, during or after the drying step.

A third aspect of the present invention provides a use of the encapsulated composition as described herein above in order to obtain a consumer product that is free of microcapsule aggregates.

Such a consumer product can comprise at least one cationic surfactant in the consumer product base. The consumer product is preferably a fabric care conditioner or a hair care conditioner.

The at least one cationic surfactant can be a quaternized triethanolamine ester selected from the group consisting of quaternized triethanolamine mono esters, quaternized triethanolamine diesters, quaternized triethanolamine triesters.

In particular embodiments, the consumer product contains at least one cationic surfactant at an amount of from 0.5 to 15 wt.-%, preferably from 1.0 wt.-% to 10 wt.-%, more preferably from 1.5 to 8.0 wt.-%, still more preferably from 2.0 to 4.0 wt.-%, based on the total weight of the consumer product.

In particular embodiments, the level of the encapsulated composition in the consumer product is from 0.01 to 5 wt.-%, preferably from 0.05 to 2.5 wt.-%, more preferably from 0.1 to 2.0 wt.-%, still more preferably from 0.1 to 1.5 wt.-%, based on the total weight of the consumer product.

Encapsulated compositions of the present invention may be employed as a delivery system to deliver active ingredients, such as perfumes for use in all manner of consumer products. The term “consumer products” refers in particular to home care, textile care or personal-care products, such as body care and hair care products.

Encapsulated compositions according to the present invention are particularly usefully employed as perfume delivery vehicles in consumer products that require, in order to deliver optimal perfumery benefits, that the microcapsules adhere well to the substrate on which they are applied. Such consumer products include hair shampoos and conditioners, as well as textile-treatment products, such as laundry detergents and conditioners.

There now follows a series of examples that serve to further illustrate the invention.

### EXAMPLES 1.1 TO 1.9: PREPARATION AND CHARACTERISTICS OF MICROCAPSULES

Polyurea microcapsules were prepared by performing the steps of (see Table 1 for details):
1) Preparing a core composition comprising 3-aminopropyltriethoxysilane by admixing a known amount of 3-aminopropyltriethoxysilane and 31 g of fragrance composition;
2) Emulsifying the core composition obtained in step 1) in 54.8 g water containing a known amount of ZeMac E400 by using a mechanical stirrer at 900 rpm at a temperature of 35±2° C.;
3) Adjusting the pH to 6.0 by addition of a 10 wt.-% solution of NaOH in water and maintaining the system stirring as in step 2) at a temperature of 35±2° C. for 1 h;
4) Adding a known amount of hydrodispersible isocyanate based on hexamethylene diisocyanate (Bayhydur XP2547, Covestro) and a known amount of diisocyanate 4,4 dicyclohexylmethanediyle (Desmodur W1, Covestro) to the emulsion and maintaining the system stirring as in steps 2) and 3) at a temperature of 35±2° C. for 30 minutes;
5) Adding a known amount of polyethyleneimine solution (Lupasol G100, BASF) in one step and heating reaction mixture gradually to 70° C. during 2 h;
6) Adding a known amount of an aqueous solution of cationic polymer (see Table 1 for details) and further heating the reaction mixture to 85° C. for 2 h;
7) Adding 1 g ammonia solution and 0.2 g of hydroxyethylcellulose (Natrosol 250HX, Ashland) and cooling down the mixture to room temperature.
8) Adjusting the final pH of the suspension to 4.0±0.2 with citric acid solution.

The volume average capsule size distribution, obtained with light scattering measurements using a Malvern 2000S instrument: The volume-median diameters D(50) and D(90) value are reported in Table 1.

The colloidal stabilitywas evaluated visually by assessing the absence of microcapsules aggregates in the samples. The formation of microcapsule aggregates is characterized by the onset of white flocs that are visible by the human eye.

TABLE 1
Formulation details and characteristics of the microcapsules (all amounts in g; n.d. means “not determined”)

| Example | 1.1 | 1.2 | 1.3 | 1.4 | 1.5 | 1.6 | 1.7 | 1.8 | 1.9 |
|---|---|---|---|---|---|---|---|---|---|
| 3-aminopropyl-triethoxysilane | 0.2 | 0.3 | 0.3 | 0.3 | 0.3 | 0.3 | 0.3 | 0.3 | 0.3 |
| ZeMac E400 | 1.7 | 0.8 | 0.8 | 0.8 | 0.8 | 0.8 | 0.8 | 0.8 | 0.8 |
| Bayhydur XP2547 | 0.2 | 0.4 | 0.4 | 0.4 | 0.4 | 0.4 | 0.4 | 0.4 | 0.4 |
| Desmodur W1 | 2.2 | 2.2 | 2.2 | 2.2 | 2.2 | 2.2 | 2.2 | 2.2 | 2.2 |
| Lupasol G100 (active) | 0.84 | 0.84 | 0.84 | 0.84 | 0.84 | 0.84 | 0.84 | 0.84 | 0.84 |
| Merquat 281; MW: 45′000 g/mol (40% | 1.25 | 4.0 | 6.0 | | | | | | |
| active material)(1) | | | | | | | | | |
| Merquat 2001; MW: 1′200′000 g/mol | | | | | | 6.4 | | | 9.6 |
| (25% active material)(2) | | | | | | | | | |
| Merquat 740; MW: 100′000 g/mol | | | | | 3.8 | | | 5.7 | |
| (42% active material)(3) | | | | | | | | | |
| Merquat 100; MW: 150′000 g/mol | | | | 4.0 | | | 6.0 | | |
| (40% active material)(4) | | | | | | | | | |
| Amine/ammonium to carboxylic acid | 0.44 | 1.03 | 1.12 | 1.53 | 0.95 | 0.68 | 1.94 | 1.05 | 0.65 |
| molar ratio | | | | | | | | | |
| D(50) [μm] | 18 | 18 | 15 | 230 | 39 | 27 | 88 | 33 | 23 |
| D(90) [μm] | 27 | 25 | 25 | 925 | 145 | 168 | 542 | 102 | 55 |
| Aggregation in softener | YES | NO | NO | n.d. | n.d. | n.d. | n.d. | NO | YES |
| Aggregation in detergent pouches | NO | NO | YES | n.d. | n.d. | n.d. | n.d. | NO | NO |
| Aggregation in liquid detergent | NO | NO | NO | n.d. | n.d. | n.d. | n.d. | NO | NO |
| Remarks | | | | Aggregates | Polydisperse | Polydisperse, | Aggregates | Polydisperse | Too |
| | | | | in | | too | in | | viscous |
| | | | | slurry | | viscous | slurry | | |

(1) Merquat 281 is a polyquaternium 22 (poly(acrylic acid-co-dimethyldiallyl ammonium chloride) copolymer), available from Merck.
(2) Merquat 100 is a polyquaternium 6 (poly(diallyldimethylammonium chloride) homopolymer), available from Merck.
(3) Merquat 740 is a polyquaternium 7 (poly(acrylamide-co-diallyldimethyl-ammonium chloride) copolymer), available from Merck.
(4) Merquat 2001 is a polyquaternium 47 (poly(acrylic acid-co-methacrylamidopropyl trimethylammonium chloride-co-methyl acrylate) copolymer), available from Merck.

As apparent from these results, microcapsules having a nominal molar ratio of amino groups and quaternary ammonium groups to carboxylic acid groups of 1.03 or 1.05, respectively, (samples 1.2 and 1.8) are colloidally stable in both cationic softener and liquid detergents. Among these samples, the one with ampholytic copolymer Merquat 281 is particularly preferred because microcapsules obtained with this copolymer are less polydisperse than the ones obtained with Merquat 740. It is anticipated that in the average molecular weight of Merquat 740 is about to exceed the range within which the desired particle size and particle polydispersity can be obtained.

Comparative example 1.1 (WO 2019/121736 A1, Example 4) has a low nominal molar ratio of the amino groups and quaternary ammonium groups to the carboxylic acid groups. The corresponding capsules are colloidally unstable in products comprising cationic surfactants.

Comparative example 1.3 has a nominal molar ratio of the amino groups and quaternary ammonium groups to the carboxylic acid groups that is slightly above the 1.1 limit. The corresponding capsules are colloidally unstable in products having both anionic surfactants and a moderately alkaline pH, such as pouch liquid detergents (pH about 8).

Comparative examples 1.4 and 1.7 have been obtained by using a cationic homopolymer. The corresponding microcapsules form aggregates in the slurry. This demonstrates that homopolymers are less favorable for the sake of the present invention than copolymer comprising co-monomers that are not cationic.

Comparative examples 1.6 and 1.9 show that Merquat 2001 increases the slurry viscosity and is therefore less suitable for the sake of the invention. Furthermore, the molar fraction of cationic moieties in this polymer (31%) is lower than the molar fraction of its carboxylic moieties (69%). Consequently, increasing the nominal molar ratio of the amino groups and/or quaternary ammonium groups to the carboxylic acid groups above 0.7 is not feasible with this polymer.

1. An encapsulated composition comprising at least one core-shell microcapsule, wherein the at least one core-shell microcapsule comprises a core containing at least one perfume ingredient and a shell surrounding the core, wherein the shell comprises a thermosetting resin formed by reaction of a polyfunctional amine comprising at least one amino group with at least one polyfunctional isocyanate,
wherein the shell further comprises a cationic polymer comprising quaternary ammonium groups,
wherein the shell further comprises a polymeric stabilizer comprising fully or partially dissociated carboxylic acid groups,
characterized in that the nominal molar ratio of the amino groups and the quaternary ammonium groups to the carboxylic acid groups is from 0.9 to 1.1.

2. The encapsulated composition according to claim 1, wherein the at least one polyfunctional isocyanate comprises an anionically modified polyisocyanate (A) selected from anionically modified hexamethylene diisocyanate, anionically modified isophorone diisocyanate, anionically modified dicyclohexylmethane-4,4′-diisocyanate, the anionically modified isocyanurate of hexamethylene diisocyanate and mixtures thereof and a non-ionic polyisocyanate (B), selected from hexamethylene diisocyanate, isophorone diisocyanate, dicyclohexylmethane-4,4′ diisocyanate, the isocyanurate of hexamethylene diisocyanate and mixtures thereof.

3. The encapsulated composition according to claim 2, wherein the weight ratio of anionically modified polyisocyanate (A) to non-ionic polyisocyanate (B) is in the range of from 0.05 to 0.5.

4. The encapsulated composition according to claim 1, wherein the polyfunctional amine comprises at least one polyethyleneimine.

5. The encapsulated composition according to claim 1, wherein the weight ratio of polyfunctional amine to polyfunctional isocyanate is from 0.05 to 1.

6. The encapsulated composition according to claim 1, wherein the polymeric stabilizer is formed by combination of a polymeric surfactant comprising the carboxylic acid groups with at least one aminosilane.

7. The encapsulated composition according to claim 1, wherein the weight ratio of the polymeric stabilizer to the thermosetting resin is from 0.4 to 0.9.

8. The encapsulated composition according to claim 1, wherein the cationic polymer is a block co-copolymer comprising quaternary ammonium groups and at least one co-monomer that is not cationic.

9. The encapsulated composition according to claim 1, wherein the cationic polymer is selected from the group consisting of polyquaternium-2 (poly(bis[2-chloroethyl]ether-alt-1,3-bis[3-(dimethylamino)propyl]urea) copolymer), polyquaternium-4 (hydroxyethyl cellulose dimethyl diallylammonium chloride copolymer), polyquaternium-5 (poly(acrylamide-b-methacrylyloxyethyltrimethyl ammonium methosulfate) copolymer), polyquaternium-6 (poly(diallyldimethylammonium chloride) homopolymer), polyquaternium-7 (poly(acrylamide-co-diallyldimethyl-ammonium chloride) copolymer), polyquaternium-9 (poly(meth-acrylyloxyethyltrimethyl ammonium bromide) homopolymer), polyquaternium-10 (hydroxyethyl cellulose 2-hydroxyethyltrimethylammonium chloride copolymer), polyquaternium-11 (poly(vinylpyrrolidone-co-dimethylaminoethyl methacrylate) copolymer), polyquaternium-12 (poly(ethylmethycralate-co-abietylmethacrylate-co-methacrylyloxyethyltrimethyl ammonium dimethylsulfate) terpolymer), polyquaternium-13 (poly(ethylmethycralate-co-oleylmethacrylate-co-methacrylyloxyethyltrimethyl ammonium dimethylsulfate) terpolymer), polyquaternium-14 (poly(methacryloyloxy)-ethyl]-N,N,N-trimethylammonium methosulfate) homopolymer), polyquaternium-15 (poly(acrylamide-co-methacrylyloxyethyltrimethyl ammonium chloride) copolymer), polyquaternium-16 (poly(vinylpyrrolidone-co-vinylimidazanium chloride) terpolymer), polyquaternium-17, (adipic acid, dimethylaminopropylamine and dichloroethylether terpolymer), polyquaternium-18 (azelaic acid, dimethylaminopropylamine and dichloroethylether terpolymer), polyquaternium-19 (quaternized copolymer of poly(vinylalcohol) and 2,3-epoxy-propylamine copolymer), polyquaternium-22 (poly(acrylic acid-co-dimethyldiallyl ammonium chloride) copolymer), polyquaternium-28 (poly(vinylpyrrolidone-co-methacylamidopropyltrimethyl ammonium chloride) copolymer), polyquaternium-29 (chitosan modified with 2,3-dihydroxypropyl-2-hydroxy-3-(trimethylammonio)propyl ether, chloride), polyquaternium-32 (poly(acrylamide-co-methacryloyloxyethyltrimethyl ammonium chloride) copolymer), polyquaternium-33 (poly(acrylamide-co-acryloyloxyethyltrimethyl ammonium chloride) copolymer), polyquaternium-34 (1,3-dibromopropane and N,N-diethyl-N′,N′-dimethyl-1,3-propanediamine copolymer), polyquaternium-35 (poly(methacryloyloxyethyltrimethylammonium-co-methacryloyloxyethyldimethylacetylammonium methosulfate) copolymer), polyquaternium-36 (poly(butyl methacrylate-co-methacryloyloxyethyldimethylamine-co-methacryloyloxyethyl-trimethylammonium dimethylsulphate) terpolymer), polyquaternium-37 (poly(methacryloyloxyethyltrimethyl ammonium chloride) homopolymer), polyquaternium-39 (poly(acrylic acid-co-acrylamide-co-diallyl-dimethylammonium chloride) terpolymer), polyquaternium-42 (poly[oxyethylene(dimethylimino)ethylene (dimethylimino)ethylene dichloride) copolymer), polyquaternium-43 (poly(acrylamide-co-acrylamidopropyltrimethyl ammonium chloride-co-2-amidopropylacrylamide sulfonate-co-dimethylaminopropylamine) copolymer), polyquaternium-44 (poly(vinylpyrrolidone-co-imidazolinium) copolymer), polyquaternium-45, (poly([N-methyl-N-ethoxyglycine]-methacrylate-co-methacryloyloxyethyl-trimethyl ammonium dimethylsulfate) copolymer), polyquaternium-46 (poly(vinylcaprolactam-co-vinylpyrrolidone-co-vinylimidazolium methosulfate) terpolymer) and polyquaternium-47 (poly(acrylic acid-co-methacrylamidopropyl trimethylammonium chloride-co-methyl acrylate) terpolymer); preferably polyquaternium-22 (poly(acrylic acid-co-dimethyldiallyl ammonium chloride) copolymer), polyquaternium-39 (poly(acrylic acid-co-acrylamide-co-diallyl-dimethylammonium chloride) terpolymer) and combinations thereof.

10. The encapsulated composition according to claim 1, wherein the level of shell material in the at least one core-shell microcapsule is from 2 wt.-% to 25 wt.-%, based on the total weight of the at least one core-shell microcapsule.

11. The encapsulated composition according to claim 1 comprising a plurality of core-shell microcapsules, wherein the volume median diameter Dv(50) of the microcapsules is from 1 to 50 μm.

12. The encapsulated composition according to claim 1 in form of a slurry, wherein the core-shell microcapsules are dispersed or suspended in an aqueous phase.

13. The encapsulated composition according to claim 12, wherein the slurry has a solid content of from 20 wt.-% to 60 wt.-%.

14. A method for obtaining the encapsulated composition according to claim 1, the method comprising the steps of:
a) Providing a core composition comprising an aminosilane;
b) Providing an aqueous phase comprising at least one polymeric surfactant comprising fully or partially dissociated carboxylic acid groups;
c) Emulsifying the core composition provided in step a) into the aqueous phase provided in step b) to obtain an emulsion of core composition droplets dispersed in the aqueous phase;
d) Causing the aminosilane and the polymeric surfactant to form a polymeric stabilizer stabilizing the dispersed oil droplets;
e) Adding at least one polyfunctional isocyanate;
f) Adding at least one polyfunctional amine comprising at least one amino group; and
g) Causing the polyfunctional isocyanate and the polyfunctional amine to react and to form a shell around the core composition droplets, in order to form a slurry of core-shell microcapsules;
wherein a cationic polymer comprising quaternary ammonium groups is added before, during or after step g); and
wherein the nominal molar ratio of the amino groups and the quaternary ammonium groups to the carboxylic acid groups is from 0.8 to 1.1.

15. A method of utilizing the encapsulated composition according to claim 1 to obtain a consumer product that is free of microcapsule aggregates.

16. A consumer product comprising the encapsulated composition according to claim 1, wherein the consumer product comprises at least one cationic surfactant in a consumer product base.

17. The consumer product according to claim 16, wherein the at least one cationic surfactant is a quaternized triethanolamine ester selected from the group consisting of quaternized triethanolamine monoesters, quaternized triethanolamine diesters, quaternized triethanolamine triesters and combinations thereof.

18. The consumer product according to claim 16, wherein the amount of the at least one cationic surfactant is from 0.5 to 15 wt.-% based on the total weight of the consumer product.

19. The consumer product according to claim 17, wherein the level of the encapsulated composition is from 0.01 to 5 wt.-%, based on the total weight of the consumer product.

20. The encapsulated composition according to claim 6, wherein the polymeric stabilizer is formed by combination of 3-aminopropyltriethoxysilane with at least one of a poly(ethylene-co-maleic anhydride) and a poly(styrene-co-maleic anhydride).
